(19) United States
(12) Patent Application Publication
(10) Pub. No.: US 2007/0156125 A1
(43) Pub. Date: Jul. 5, 2007

(54) Encodable cryogenic device

(76) Inventor: Russell DeLonzor, San Ramon, CA (US)

Correspondence Address: SULLIVAN & WORCESTER LLP, ONE POST OFFICE SQUARE, BOSTON, MA, 02109, US

(21) Appl. No.: 11/324,178

(22) Filed: Dec. 30, 2005

(51) Int. Cl. A61B 18/02; A61B 17/00

(52) U.S. Cl. 606/21; 606/20; 606/1

(57) ABSTRACT

A medical device, such as a cryogenic biopsy device, includes a disposable unit and a reusable unit. The disposable unit, such as a treatment needle or probe, is at least partially insertable into the body of a medical subject and includes an encodable device for storing information. The reusable unit is connectable to the disposable unit and includes a controller. The disposable unit transfers the information to the controller in the reusable unit. The controller then directs activities of the medical device in response to the information stored in the disposable unit.

### CROSS-REFERENCES TO RELATED APPLICATIONS

The following patents and/or patent applications are hereby incorporated by reference herein in their entirety:

U.S. Pat. No. 6,551,255, entitled “Device for Biopsy of Tumors,” filed Oct. 16, 2000; U.S. patent application Ser. No. 10/421,598, entitled “Device for Biopsy of Tumors,” filed Apr. 22, 2003; U.S. Pat. No. 6,789,545, entitled “Method and System for Cryoablating Fibroadenomas,” filed Oct. 4, 2002; and U.S. patent application Ser. No. 10/941,511, entitled “Method and System for Cryoablating Fibroadenomas,” filed Sep. 14, 2004. U.S. patent application Ser. No. 11/210,436, entitled “Rotational Core Biopsy Device with Liquid Cryogen Adhesion Probe,” filed Aug. 23, 2005.

### STATEMENT REGARDING FEDERALLY SPONSORED RESEARCH OR DEVELOPMENT

Not Applicable.

### REFERENCE TO MICROFICHE APPENDIX

Not Applicable

### BACKGROUND OF THE INVENTION

1. Field of the Invention

This invention relates generally to medical devices, and, more particularly, to electronically controlled medical devices used to conduct procedures that involve inserting at least part of a sterile object into the body of a medical subject.

2. Description of Related Art

Many medical procedures involve inserting a sterile object, such as a needle or probe, into the body of a medical subject, such as a human patient. For instance, certain biopsy procedures involve inserting a needle into a patient and removing a portion of suspect tissue.

Medical devices for performing these types of procedures can be complex. One such device is described in U.S. Pat. No. 6,551,255. As described therein, a biopsy device includes a source of cryogenic fluid, a mechanism for releasing the cryogenic fluid, a needle for conducting the cryogenic fluid toward an administration site, a cutting cannula for cutting a core of tissue surrounding the needle, and a drive system for advancing and retracting the cannula.

The design of this biopsy device has evolved over time to become more automated. It has been equipped with a user console, a power source, and a microprocessor. These features allow a biopsy procedure to be conducted largely under electronic control. The microprocessor responds to user button-presses at the console to perform sequences of activities. For example, once a user inserts a biopsy needle into a suspect mass, the user presses a button. The device then automatically performs a sequence of steps, which include administering a designated amount of cryogenic fluid, waiting a designated period of time for tissue to adhere to the needle, and advancing the cutting cannula after the designated period of time expires.

As this biopsy device has become more complex, it has also become more expensive. Expense is an important factor to consumers of these devices because each device can generally be used only once. After its first use, the device is no longer sterile and is generally discarded.

To avoid discarding an entire device after a single use, the device has been modified to be more modular. Reusable portions of the device have been segregated from disposable, single-use portions. The disposable parts of the device include the needle and the cutting cannula, i.e., the portions that are actually insertable into the body of a patient. Other portions of the device are reusable. An example of this type of device is disclosed in U.S. patent application Ser. No. 11/210,436, entitled “Rotational Core Biopsy Device with Liquid Cryogen Adhesion Probe.”

As is known, different biopsy procedures call for needles and cannulas of different gauges, lengths, and/or compositions. There are many reasons for this. One is the type of mass being biopsied—whether it is soft or hard, mobile or immobile, densely or sparsely vascular. Another is the location of the mass within the patient's body—whether it is close to the surface or deep. Yet another is the context in which the device is to be used. For example, ferrous needles and cannulas should generally be avoided for procedures guided by magnetic resonance imaging (MRI).

For optimal results, the conduct of a biopsy procedure is preferably varied to account for differences in the gauge, length, and/or composition of the needle and/or cannula used. For instance, greater amounts of cryogenic fluid may be needed for larger needles. Longer delays may be needed between administering the cryogenic fluid and advancing the cannula for larger cannulas.

Previously, the need for different needles and cannulas has been managed by providing integrated biopsy devices specifically tailored for different applications. Each device was programmed to perform optimally with the needle and cannula that it included.

In making the design more modular, however, it has become desirable to allow the reusable portion of the device to be mated with a wide variety of needles and cannulas. This gives rise to a new problem, however: how to ensure that optimal settings are used for the selected needle and/or cannula. What is needed is an effective way of varying the settings of the reusable portion of a device depending upon the particular disposable parts used.

### SUMMARY

According to an embodiment of the invention, a medical device for performing a medical procedure includes a disposable unit and a reusable unit. The disposable unit is at least partially insertable into the body of a medical subject and includes an encodable device for storing information. The reusable unit is connectable to the disposable unit and includes a controller for directing actions of the medical device in response to the information stored in the disposable unit.

According to another embodiment, a medical device includes a housing and a mechanical interface portion. The mechanical interface portion is disposed at least partially within or upon the housing and is adapted for mechanically engaging with a disposable unit. The disposable unit is at least partially insertable into a medical subject and stores information pertinent to the medical procedure. A controller is disposed within the housing for conducting automated steps of the medical procedure. These steps are performed in response to information received from the disposable unit.

According to a further embodiment, a medical device for performing a medical procedure includes a disposable unit that is insertable into a medical subject. The disposable unit has an encodable device for storing information and an interface portion for mechanically engaging with a reusable unit.

According to yet another embodiment, a medical device includes a plurality of disposable units and a reusable unit. Each disposable unit is insertable into the body of a medical subject and includes an encodable device for storing information. The reusable unit includes a controller for directing automated actions to be performed by the medical device in response to the information stored in the encodable device. The reusable unit has a mechanical interface portion for mechanically engaging with a respective disposable unit and a communications interface portion for receiving the information from the respective disposable unit.

According to another embodiment, a method for performing a medical procedure includes engaging a first unit with a second unit. The method further includes conveying information stored in the first unit to the second unit, inserting at least part of the first unit into the body of a medical subject, and performing a medical procedure on the medical subject in response to the copied information.

According to yet another embodiment, a method for performing a medical procedure includes engaging a first unit with a second unit. The first unit is insertable into the body of a medical subject. The method further includes conveying information stored in the first unit to the second unit and configuring the second unit for performing automated portions of the medical procedure responsive to the information copied from the first unit.

### BRIEF DESCRIPTION OF THE DRAWINGS

Additional objects, advantages, and novel features of the invention will become apparent from a consideration of the ensuing description and drawings, in which

FIG. 1 is a side perspective view of a modular biopsy device according to an embodiment of the invention;

FIG. 2 is a side perspective view of the modular biopsy device of FIG. 1 separated into a reusable portion and a disposable portion;

FIG. 2A is a rear plan view of the disposable portion of the modular biopsy device of FIG. 1;

FIG. 3 is a block diagram of an embodiment of an encodable device used with the disposable portion of the biopsy device of FIG. 1;

FIG. 4 is a block diagram of an embodiment of electronic circuitry included within a reusable portion of the biopsy device of FIGS. 1-2;

FIG. 5 is a flowchart of a process for configuring a reusable portion of a medical device based on information contained in a disposable portion of the medical device;

FIG. 6 is a front view of a modular treatment device according to an embodiment of the invention;

FIG. 7 is a front view of the modular treatment device of FIG. 5 separated into a reusable portion and a disposable portion;

FIG. 8 is a block diagram of an embodiment of electronic circuitry included within a reusable portion of the treatment device of FIGS. 6 and 7; and

FIGS. 9-10 are block diagrams of alternative implementations of encodable devices that may be used with the disposable portions of the biopsy device of FIGS. 1-2 or the treatment device of FIGS. 6-7.

### DETAILED DESCRIPTION OF THE PREFERRED EMBODIMENT

As used throughout this document, the words “comprising,” “including,” and “having” are intended to set forth certain items, steps, elements, or aspects of something in an open-ended fashion. Unless a specific statement is made to the contrary, these words do not indicate a closed list to which additional things cannot be added.

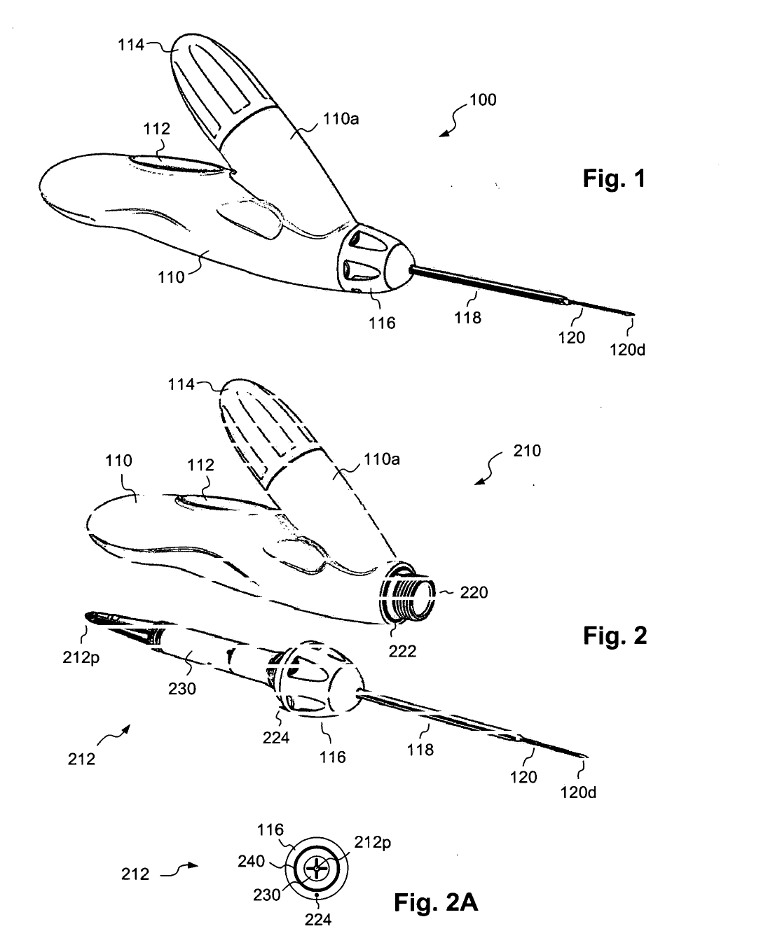

FIGS. 1 and 2 show an example of a medical device 100 according to an embodiment of the invention. The medical device 100 is adapted for acquiring biopsy samples of suspect masses, such as fibroadenomas. The device 100 includes a housing 110, a user console 112, a knob portion 114, and a nose portion 116. A cutting cannula 118 and a biopsy needle 120 extend from the nose portion 116. The biopsy needle 120 has a sharp distal tip 120d and is disposed coaxially within the cutting cannula 118. The cutting cannula 118 is both translatable along the axis of the biopsy needle 120 and rotatable about the axis of the biopsy needle 120.

The nose portion 116 is threadedly attached the housing 110. By unscrewing the nose portion 116 from the housing 110, the device 100 can be separated into two distinct portions: a reusable portion 210 and a disposable portion 212. A threaded region 220 extends from the housing 110 of the reusable portion 210 and mates with a complementarily threaded region 240 (See FIG. 2A) at the rear of the nose 116 of the disposable portion 212.

The reusable portion 210 includes components that may be used repeatedly for performing a large number of biopsy procedures. These include the housing 110, user console 112, and knob 114. The reusable portion 210 also includes internal components (not shown), such as a battery, an electronic controller, a motor and gearbox, and various pneumatic tubes and valves. The knob 114 can be unscrewed for inserting a canister of compressed gas, such as CO₂ or N₂O, which provides both a source of cryogenic fluid and a source of pneumatic pressure.

The disposable portion 212 is preferably used only once. It includes the nose portion 116, the cutting cannula 118, and the biopsy needle 120. The disposable portion 212 also includes a rear portion 230, which fits within the housing 110 of the reusable portion 210. The rear portion 230 includes a coring mechanism (not shown) for both translating and rotating the cutting cannula 118 with respect to the biopsy needle 120. The reusable portion 212 has a proximal end 212p, which is adapted for receiving compressed gas from the reusable unit 210.

A biopsy device substantially as described above is disclosed in U.S. patent application Ser. No. 11/210,436, entitled “Rotational Core Biopsy Device With Liquid Cryogen Adhesion Probe,” which is hereby incorporated by reference.

In contrast with prior devices, however, the device 100 includes an encodable device, housed within the disposable portion 212, for storing information. In the example shown in FIGS. 1 and 2, the encodable device is preferably an electronic circuit encapsulated within the nose portion 116. When the disposable portion 212 is attached to the reusable portion 210, a circuit is formed between the disposable portion 212 and the reusable portion 210.

Preferably, each of the threaded regions 220 and 240 is composed of or includes a conductive material. An electrical contact, such as a ring 222 of conductive material, is preferably disposed at the front of the housing 110 proximate to the threaded region 220 and electrically insulated from the threaded region 220. An electrical contact, such as a spring-loaded pin 224, is preferably disposed at the rear of the nose 116 and is electrically insulated from the conductive material within the threaded region 240.

Both the pin 224 and the conductive material of the threaded region 240 are electrically connected to the encodable device. In addition, both the ring 222 and the conductive material on the threaded region 220 are electrically connected to the controller within the housing 110. The encodable device thus forms an electrical circuit with the controller when the disposable portion 212 is attached to the reusable portion 210.

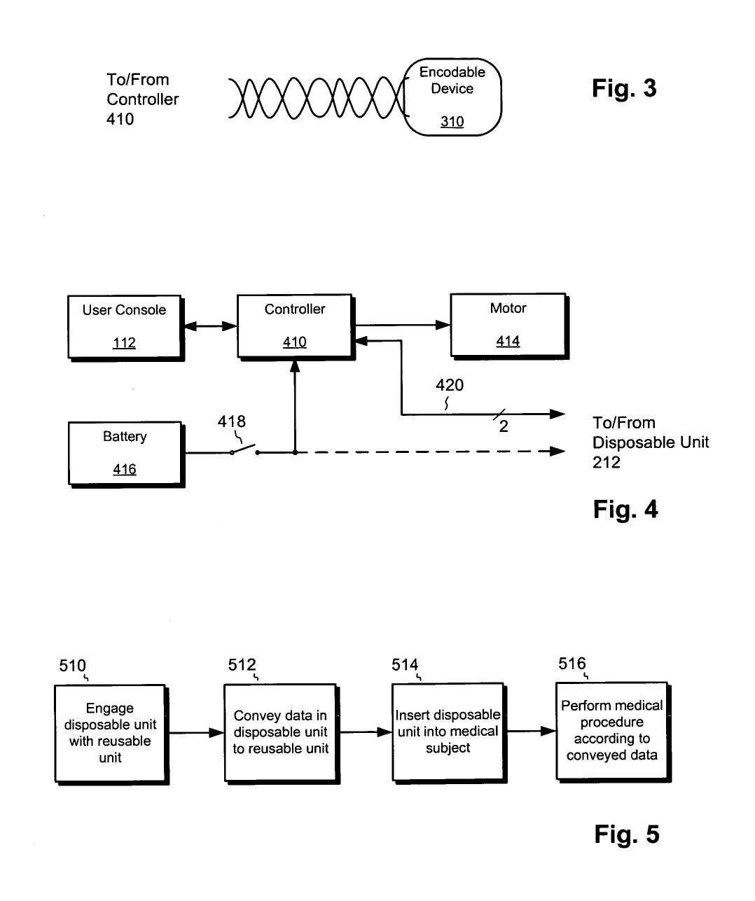

FIG. 3 shows the encodable device 310. The encodable device 310 is preferably encapsulated within the nose portion 116 of the disposable portion 212. The device 310 preferably electrically interfaces with its environment via two conductors. One conductor, such as a wire, is run from one terminal of the device 310 to the pin 224. Another conductor, such as a wire, is run from another terminal of the device 310 to the threaded region 240.

The encodable device is preferably a “1-wire®” nonvolatile memory circuit, such as a DS28E04-100 available off-the-shelf from Dallas Semiconductor Corp. of Dallas, Tex. The DS28E04-100 includes a 4 kb EEPROM (Electronically Erasable Programmable Read-Only Memory). As is known, “1-wire” circuits are able to manage serial communications with their environments and receive power from their environment via only two conductors. These circuits are thus well suited for compact implementations in which it is desirable to minimize the number of electrical interconnections.

FIG. 4 shows an example of electronic circuitry disposed within the housing 110 of the reusable portion 210. The circuitry includes a controller 410. The controller preferably includes a microprocessor. The controller 410 is operatively connected to the user console 112, for receiving user input, in the form of button presses, and for illuminating various indicators for displaying status. The controller 410 is also operatively connected to a motor 414, for controlling the application of pneumatic pressure to various parts of the device 100. A power source, such as a battery 416, provides electrical power, via a switch 418, for operating the controller 410, the user console 112, and the motor 414. The switch 418 is preferably integrated with the knob portion 114 (see FIG. 1) in a manner that causes the switch 418 to be closed when the knob 114 is turned.

The controller 410 preferably establishes a connection to the encodable device 310 via a pair of conductors, such as wires 420. One of the wires 420 is connected between the controller 410 and the conductive material of the threaded region 220. The other of the wires 420 is connected between the controller 410 and the conductive ring 222. Connection between the controller 410 and the encodable device 310 is made when the disposable portion 212 is attached to the reusable portion 210.

FIG. 5 shows a process in which a medical device can be used in accordance with the invention. At step 510, a disposable unit is engaged with a reusable unit. The disposable unit stores information pertinent to the conduct of the medical procedure. At step 512, the information, or “data,” stored in the disposable unit is conveyed to the reusable unit. At step 514, the disposable unit, or a portion thereof, is inserted into a medical subject. Certain steps of the medical procedure are then performed in response to the data conveyed (step 516).

Applying this process in the context of the device 100, the disposable portion 212 is engaged with the reusable portion 110. Electrical, mechanical, and pneumatic connections between the disposable and reusable portions are made. Data stored in the encodable device 310 is then copied to the controller 410. An incision is made in a medical subject in the vicinity of a suspect mass, such as a fibroadenoma. The biopsy needle 120 is inserted into the suspect mass, generally under ultrasound or MRI guidance. The user then operates a control on the user console 112 to initiate certain automated aspects of the process. The controller 410 responds to the user control by executing a series of actions. These actions are based, in whole or in part, on the data conveyed from the encodable device 310.

The encodable device 310 can be made to store a wide range of data pertinent to the conduct of the biopsy procedure. For example, the encodable device 310 may store the gauge (i.e., diameter), length, composition, and cooling power of the biopsy needle 120, as well as the gauge, length, and composition of the cutting cannula 118. The encodable device 310 can be made to store an indication of whether the biopsy needle 120 and/or cutting cannula 118 are MRI-compatible, e.g., whether they contain any ferrous material. The encodable device 310 can also store information about the mass being biopsied, and how much cooling is required with that mass to obtain an adequate sample.

According to one variant, the encodable device 310 stores parameters for conducting a biopsy procedure. The controller 410 reads the parameters and adjusts its activities accordingly. For example, one parameter can describe the period of time over which cryogenic fluid is conducted to the biopsy needle 120 before the cannula 118 is advanced. The controller 410 responds to this parameter by timing the application of cryogenic fluid and advancing the cannula when the desired time limit is reached.

Another parameter may describe a desired temperature that the biopsy needle 120 should attain before the cutting cannula 118 is advanced. In this instance, the biopsy needle 120 is equipped with a temperature measuring device, such as a thermocouple, which is electrically connected back to the controller 410. The controller responds to this parameter by monitoring the temperature of the biopsy needle 120 and advancing the cannula 118 when the measured temperature reaches the desired temperature.

According to another variant, the encodable device 310 stores code for conducting all automated portions of the biopsy procedure. The code can be in the form of object code directly readable by the controller 410. Rather than simply specifying parameters, an entire program is uploaded to the controller. The controller 410 then executes the code to conduct all of the functions associated with cooling the biopsy needle 120 and advancing the cannula 118.

In the examples given above, the data stored in the encodable device 310 is used for controlling the operation of the biopsy device 100. Data can be stored in the device 310 for other purposes, as well. An adapter (not shown) can be provided for connecting a disposable portion 212 of the device 100 to a computer. The computer can then read and display the data stored in the encodable device 310. This data may include, for example, the name of the patient for whom the disposable portion 212 is intended, or for whom it was used. It may include portions of the patient's medical history, or special instructions for conducting the biopsy procedure.

The encodable device 310 is preferably programmed with appropriate parameters, code, or other information, in the factory where it is assembled. However, the device 310 is also preferably programmable by the user. The computer adapter, described above, preferably allows a user to both read from and write to the device 310. A user may thus modify parameters or store additional information in the encodable device 310 prior to performing the medical procedure.

The controller 414 can preferably write to the encodable device 310 in situ, while the reusable and disposable portions are mated together. The controller 414 preferably monitors activities of the biopsy device 100 during each biopsy procedure, and stores diagnostic information related to any errors or anomalies in the encodable device 310. A user can access this diagnostic information after the biopsy procedure to explore the nature of the error or anomaly.

To prevent unauthorized access, the data stored in the encodable device 310 is preferably encrypted. The controller 414 is provided with an encryption key for decoding data received from the encodable device 310.

The biopsy device 100 offers numerous benefits. Disposable portions having needles and cannulas of different gauges, lengths, and compositions can be used with a single reusable portion. Settings for performing the biopsy with the selected probe needle and cannula are automatically adjusted to optimal values.

Alternatives

Having described one embodiment, numerous alternative embodiments or variations can be made. For instance, the invention is not limited to a biopsy device.

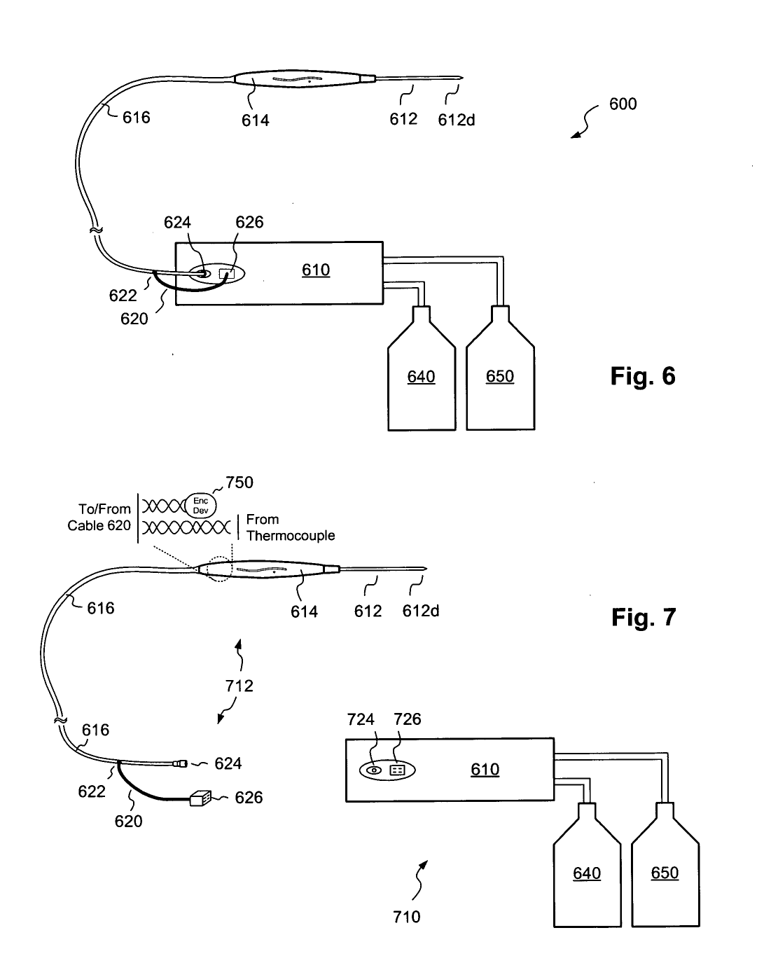

FIGS. 6 and 7 show an example of a cryogenic treatment device 600 according to an alternative embodiment of the invention. Like the biopsy device 100, the treatment device 600 is separable into a reusable portion 710 and a disposable portion 712.

The reusable portion 710 includes a control unit 610. The control unit 610 is preferably plumbed to two cryogenic sources, a first tank 640 containing compressed Argon gas and a second tank 650 containing compressed Helium gas. The control unit 610 has pneumatic connector 724 and an electrical connector 726. The control unit preferably includes electronic and pneumatic components for controlling the application of pressurized Argon and Helium gas to the connector 724. It also includes electronic components for measuring the temperature of the thermocouple by measuring a voltage applied to two terminals of the connector 726.

The disposable portion 712 includes a hand-held unit 614, a hollow treatment needle 612, and a flexible tube 616. The flexible tube 616 encloses a pneumatic tube (not shown), which extends from a distal tip 612d of the treatment needle 612 to a pneumatic connector 624. The flexible tube 616 also encloses an electrical cable 620, which extends from a thermocouple (not shown) attached within the treatment needle 612 to an electrical connector 626. The electrical cable 620 preferably emerges from the flexible tube 616 via a hole 622 in the flexible tube 616.

During operation, the connectors 624 and 626 are respectively mated with the connectors 724 and 726. An incision is made in a medical subject, in a vicinity of a mass to be treated, and the treatment needle 612 is inserted into the mass, generally under ultrasound or MRI guidance. A user then operates one or more controls on the control unit 610. In response, the control unit 610 performs a sequence of timed actions, such as applying pressurized Argon or Helium gas to the disposable portion. These actions may be adjusted based on temperature readings from the thermocouple.

A treatment device substantially as described above is disclosed in U.S. Pat. No. 6,789,545, entitled, “Method and System for Cryoablating Fibroadenomas,” which is hereby incorporated by reference.

In contrast with the prior device, however, the treatment device 600 includes an encodable device 750 for storing information. The encodable device 750 is preferably housed within the hand-held unit 614 and is wired back to the control unit 610 via the cable 620 and connector 626. The encodable device 750 is preferably of the same type as that described above, i.e., a “1-Wire” DS28E04-100, from Dallas Semiconductor. To accommodate the encodable device, the treatment device disclosed in U.S. Pat. No. 6,789,454 is modified. The cable 620 is modified to carry four wires instead of the two previously needed for the thermocouple, and the connectors 626 and 726 are changed to four conductor connectors. Changes are also made within the control unit 610 to accommodate the additional wiring and functionality.

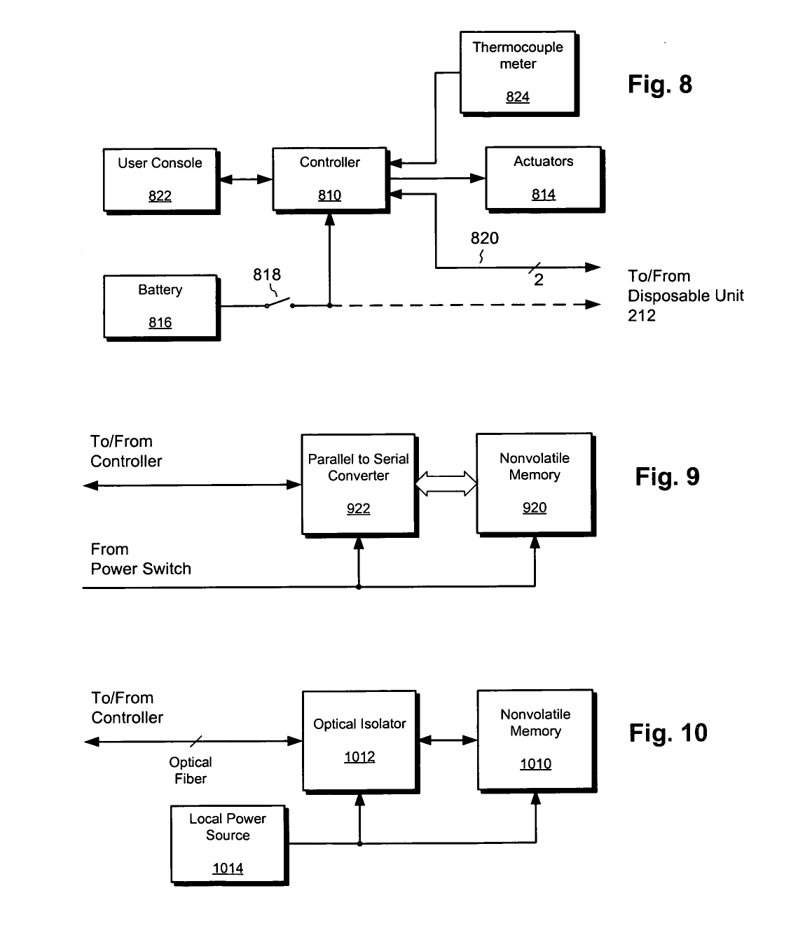

FIG. 8 shows an example of electronic circuitry disposed within the control unit 610 of the reusable portion 710. The control unit 610 includes an electronic controller 810, which preferably includes a microprocessor. The controller 810 is coupled to a user console 822, for receiving commands from a user and providing status to the user. The controller 810 is coupled to a thermocouple meter 824, for measuring a voltage generated by the thermocouple (via the connector 726). It is also coupled to one or more actuators 814, for controlling the application of Argon and Helium to the pneumatic connector 724. A power source, such as a battery 816, provides electrical power, via a switch 818, for operating the controller 810, the user console 822, the actuators 814, and the thermocouple meter 824.

The controller 810 preferably establishes a connection to the encodable device 750 via a pair of conductors, such as wires 820, which are routed to pins of the connector 726. Connections between the controller 810 and the encodable device 750 are made when the disposable portion 712 is attached to the reusable portion 710.

As with the biopsy device 100, optimal conduct of a medical procedure involving the treatment device 600 depends upon proper settings. For example, certain treatment settings are optimally set in response to the gauge, length, and/or composition of the treatment needle, the cooling power of the treatment needle, and the shape of the treatment zone, as well as other factors. These settings describe, for example, the amount of cryogenic fluid to be applied, the sequence of fluid applications, and the timing between fluid applications, all of which are controlled by the (reusable) control unit 610.

The encodable device 750 preferably stores these settings. As described above in connection with the biopsy device 100, these settings may be stored as parameters that the controller 810 reads and applies for adjusting the conduct of a treatment procedure. Alternatively, an entire code section or program for conducting the medical procedure can be stored on the encodable device 750. The code is copied to the controller 810, and the controller runs the program.

The invention is not limited to cryogenic biopsy and treatment devices. It may be used in connection with any type of medical device that includes disposable and reusable portions.

As described herein, the encodable device is preferably a 1-wire device that both receives power and communicates with its environment via two conductors. This is not required, however. The encodable device can be any type of device, component, or assembly, that stores information that is readable by the reusable portion of the device.

FIG. 9 shows an alternative implementation of the encodable device. Here, a nonvolatile memory circuit 920 is used in connection with a parallel-to-serial converter 922. The parallel-to-serial converter 922 communicates serially with the controller 410/810 in the reusable portion 110/710, but communicates with the nonvolatile memory circuit 920 using parallel data. Power is supplied from the reusable portion.

FIG. 10 shows another implementation of the encodable device. Here, a nonvolatile memory circuit 1010 is coupled to an optical isolator for communicating with the controller on the reusable portion via one or more optical fibers. A local power source 1014 is included with the disposable portion to power the nonvolatile memory circuit 1010 and the optical isolator 1012. If the memory circuit 1010 operates with parallel data, a parallel-to-serial converter, like the one shown in FIG. 9, may be included between the optical isolator 1012 and the memory circuit 1010. Communication to the reusable portion can then be achieved using a single optical fiber. This implementation may be preferred in applications where it is necessary to maintain strict electrical isolation between the medical device and the patient.

Another implementation of the encodable device is an RFID (Radio Frequency Identification) device. The RFID device is made to store information, such as one or more parameters associated with the disposable portion of the device. The reusable portion would then include an RF port for reading the RFID device. When the disposable portion is brought within close proximity of the reusable portion, the RF port is directed to read the information.

The encodable device may also be implemented as an optically readable code, such as one or more barcodes. The reusable portion can be equipped with an optical reader, such as a barcode reader. By sweeping the barcode(s) with the barcode reader, the information encoded in the barcode(s) is then transferred to the reusable portion to be used in conducting a medical procedure.

A very simple implementation of the encodable device is a resistor. The reusable portion of a medical device can be equipped with a resistance measuring device, such as an ohmmeter. Resistors having different resistances could thus be made to indicate different parameters or groups of parameters. For instance, a 3 kilo-ohm resistor could indicate a 3 gauge needle, whereas a 5 kilo-ohm resistor could indicate a 5 gauge needle. Arrays or circuits of resistors or other analog components can be used to store information, as can arrays or circuits of digital components.

As shown and described, electrical connections between the reusable and disposable portions of the devices 100 and 600 are made using two conductors. Alternatively, more conductors may be used, such as for conveying digital signals in parallel form. In addition, the mechanical, electrical and pneumatic connections between the reusable and disposable portions may be integrated together, as they are for the biopsy device 100. Alternatively, they may be separated, as are the electrical and pneumatic connections for the treatment device 600.

Those skilled in the art will therefore understand that various changes in form and detail may be made to the embodiments disclosed herein without departing from the scope of the invention and appended claims.

What is claimed is:

1. A medical device for performing a medical procedure, comprising:
a disposable unit at least partially insertable into the body of a medical subject and including an encodable device for storing information;
a reusable unit connectable to the disposable unit and including a controller for directing actions of the medical device responsive to the information stored in the disposable unit.

2. A medical device as recited in claim 1, wherein the encodable device comprises an electronic circuit.

3. A medical device as recited in claim 2, wherein the reusable unit comprises a power source for applying power to the electronic circuit responsive to the reusable unit being connected to the disposable unit.

4. A medical device as recited in claim 3, wherein a communication pathway is established between the reusable unit and the disposable unit responsive to the reusable unit being connected to the disposable unit.

5. A medical device as recited in claim 4, wherein the reusable unit is electrically connected to the disposable unit with no more than two conductors when the reusable unit is connected to the disposable unit.

6. A medical device as recited in claim 5, wherein the electronic circuit comprises a device that both receives power and communicates via two conductors.

7. A medical device as recited in claim 6, wherein the device comprises a non-volatile memory.

8. A medical device as recited in claim 2, wherein the electronic circuit further comprises a serial communication interface.

9. A medical device as recited in claim 2, wherein the electronic circuit comprises non-volatile memory.

10. A medical device as recited in claim 9, wherein the non-volatile memory comprises an EEPROM.

11. A medical device as recited in claim 2, wherein the encodable device comprises an array of analog components arranged for storing the information.

12. A medical device as recited in claim 2, wherein the encodable device comprises an analog circuit arranged for storing the information.

13. A medical device as recited in claim 2, wherein the encodable device comprises at least one analog component arranged for storing the information.

14. A medical device as recited in claim 2, wherein the encodable device comprises at least one digital component for storing the information.

15. A medical device as recited in claim 1, wherein the encodable device comprises at least one optically encodable device for storing the information.

16. A medical device as recited in claim 1, wherein the disposable unit comprises a passageway for conducting a cryogenic fluid into the medical subject.

17. A medical device as recited in claim 1, wherein the encodable device is both readable and writable.

18. A medical device as recited in claim 17, wherein the encodable device is writable for storing diagnostic information produced during the medical procedure.

19. A medical device as recited in claim 1,
wherein the reusable unit comprises a processor for reading the information from the disposable unit and controlling automated aspects of the medical procedure.

20. A medical device as recited in claim 19,
wherein the information stored in the disposable unit comprise parameters for applying a cryogenic fluid to the medical subject, and
wherein the processor is constructed and arranged for adjusting an application of the cryogenic fluid responsive to the parameters.

21. A medical device as recited in claim 19,
wherein the information stored in the disposable unit comprise parameters for performing automated mechanical processes associated with the medical procedure, and
wherein the processor is constructed and arranged for adjusting the mechanical processes responsive to the parameters for performing the mechanical processes.

22. A medical device as recited in claim 19,
wherein the information stored in the disposable unit comprise information indicative of whether the disposable unit is suitable for use with an MRI procedure.

23. A medical device as recited in claim 19, wherein the disposable unit further comprises:
a needle having a distal tip; and
a passage within the needle for conducting a cryogenic material toward the distal tip.

24. A medical device as recited in claim 23, wherein the needle has a diameter, and wherein the information stored in the disposable unit comprise information indicative of a diameter of the needle.

25. A medical device as recited in claim 23, wherein the disposable unit further comprises a substantially cylindrical cutting cannula disposed coaxially around the needle and being movable along an axis of the needle.

26. A medical device as recited in claim 25, wherein the cutting cannula has a diameter, and the information stored in the disposable unit comprise information indicative of the diameter of the cutting cannula.

27. A medical device for performing a medical procedure, comprising:
a housing;
a mechanical interface portion, disposed at least partially within or upon the housing and adapted for engaging a disposable unit;
a communications interface portion for receiving information stored in the disposable unit; and
a controller, disposed within the housing, for conducting automated steps of the medical procedure responsive to the information received from the disposable unit;

28. A medical device as recited in claim 27, wherein the communications interface portion comprises conductors of electronic signals.

29. A medical device as recited in claim 27, wherein the communications interface portion comprises at least one conductor of optical signals.

30. A medical device as recited in claim 27, wherein the communications interface portion comprises at least one conductor of radiofrequency signals.

31. A medical device as recited in claim 27, wherein the mechanical interface portion comprises the communications interface portion.

32. A medical device as recited in claim 31, wherein the communications interface portion comprises conductors of electronic signals.

33. A medical device as recited in claim 31, wherein the communications interface portion comprises at least one conductor of optical signals.

34. A medical device as recited in claim 27, further comprising a fluid interface portion disposed at least partially within the housing and adapted for engaging with a fluid interface portion of the disposable unit, for conducting a cryogenic fluid to the disposable unit.

35. A medical device as recited in claim 34, wherein the mechanical interface portion comprises a communications interface portion for receiving the information stored in the disposable unit.

36. A medical device as recited in claim 27, further comprising a passageway for administering a cryogenic fluid to the disposable unit.

37. A medical device as recited in claim 36, further comprising a reservoir of cryogenic fluid.

38. A medical device as recited in claim 36, wherein the controller comprises a program for applying the cryogenic fluid to the medical subject.

39. A medical device for performing a medical procedure, comprising:
a disposable unit that is insertable into a medical subject, the disposable unit having an encodable device for storing information and a mechanical interface portion for engaging with a reusable unit.

40. A medical device as recited in claim 39, further comprising a communications interface portion for providing the information stored in the disposable unit to the reusable unit.

41. A medical device as recited in claim 40, wherein the communications interface portion comprises conductors of electronic signals.

42. A medical device as recited in claim 41, wherein the communications interface portion comprises no more than two conductors of electronic signals.

43. A medical device as recited in claim 40, wherein the communications interface portion comprises at least one conductor of optical signals.

44. A medical device as recited in claim 40, wherein the communications interface portion comprises at least one conductor of radiofrequency signals.

45. A medical device as recited in claim 39, wherein the mechanical interface portion comprises a communications interface portion for providing the information stored in the disposable unit to the reusable unit.

46. A medical device as recited in claim 45, wherein the communications interface portion comprises conductors of electronic signals.

47. A medical device as recited in claim 46, wherein the communications interface portion comprises no more than two conductors of electronic signals.

48. A medical device as recited in claim 45, wherein the communications interface portion comprises at least one conductor of optical signals.

49. A medical device as recited in claim 39, further comprising a fluid interface portion adapted for engaging with a fluid interface portion of the reusable unit, for receiving a cryogenic fluid from the disposable unit.

50. A medical device as recited in claim 39, wherein the mechanical interface portion comprises a fluid interface portion adapted for engaging with a fluid interface portion of the reusable unit, for receiving a cryogenic fluid from the reusable unit.

51. A medical device as recited in claim 50, wherein the mechanical interface portion comprises a communications interface portion for providing the information to the reusable unit.

52. A medical device as recited in claim 39, further comprising a passageway for receiving a cryogenic fluid from the reusable unit.

53. A medical device as recited in claim 39, wherein the encodable device comprises an electronic circuit.

54. A medical device as recited in claim 53, wherein the electronic circuit receives power from the reusable unit when the disposable unit is connected to the reusable unit.

55. A medical device as recited in claim 54, wherein the electronic circuit comprises a device that both receives power and communicates via two conductors.

56. A medical device as recited in claim 55, wherein the device comprises a non-volatile memory.

57. A medical device as recited in claim 53, wherein the electronic circuit further comprises a serial communication interface.

58. A medical device as recited in claim 53, wherein the electronic circuit comprises non-volatile memory.

59. A medical device as recited in claim 58, wherein the non-volatile memory comprises an EEPROM.

60. A medical device as recited in claim 39, wherein the encodable device comprises an array of analog components for storing the information.

61. A medical device as recited in claim 39, wherein the encodable device comprises at least one digital component for storing the information.

62. A medical device as recited in claim 39, wherein the encodable device comprises at least one optically encodable device for storing the information.

63. A medical device as recited in claim 39, wherein the information stored in the disposable unit comprise parameters for applying a cryogenic fluid to the medical subject.

64. A medical device as recited in claim 39, wherein the information stored in the disposable unit comprise parameters for performing automated mechanical processes associated with the medical procedure.

65. A medical device as recited in claim 39, wherein the information stored in the disposable unit comprise information indicative of whether the disposable unit is suitable for use with an MRI procedure.

66. A medical device as recited in claim 39, wherein the disposable unit further comprises:
a needle having a distal tip; and
a passage within the needle for conducting a flow of cryogenic material toward the distal tip.

67. A medical device as recited in claim 66, wherein the needle has a diameter, and wherein the information stored in the disposable unit comprise information indicative of the diameter of the needle.

68. A medical device as recited in claim 67, wherein the disposable unit further comprises a substantially cylindrical cutting cannula disposed concentrically around the needle and being movable along an axis of the needle alternatively toward the distal tip of the needle and away from the distal tip of the needle.

69. A medical device as recited in claim 68, wherein the cutting cannula has a diameter, and the information stored in the disposable unit comprise information indicative of the diameter of the cutting cannula.

70. A medical device as recited in claim 39, wherein the encodable device is both readable and writable.

71. A medical device as recited in claim 70, wherein the encodable device is writable for storing diagnostic information produced during the medical procedure.

72. A medical device for performing a medical procedure, comprising:
a plurality of disposable units, each disposable unit being insertable into the body of a medical subject and including an encodable device for storing information;
a reusable unit including a controller for directing automated actions to be performed by the medical device responsive to the information stored in the encodable device,
wherein the reusable unit has a mechanical interface portion for mechanically engaging with a respective disposable unit and a communications interface portion for receiving the information from the respective disposable unit.

73. A medical device as recited in claim 72, wherein the reusable unit further has a cryogenic interface portion for conducting a cryogenic fluid into the respective disposable unit.

74. A medical device as recited in claim 72, wherein the mechanical interface portion and the communications interface portion are physically integrated.

75. A medical device as recited in claim 73, wherein the mechanical interface portion and the cryogenic interface portion are physically integrated.

76. A medical device as recited in claim 73, wherein the mechanical interface portion, the communications interface portion, and the cryogenic interface portion are physically integrated.

77. A method for performing a medical procedure, comprising:
engaging a first unit with a second unit;
conveying information stored in the first unit to the second unit;
inserting at least part of the first unit into the body of a medical subject; and
performing a medical procedure on the medical subject responsive to the copied information.

78. A method as recited in claim 77, wherein the step of performing the medical procedure comprises conducting a cryogenic fluid from the second unit to the first unit.

79. A method as recited in claim 78,
wherein the information stores at least one parameter indicative of a predetermined amount of time that the cryogenic fluid is conducted from the second unit to the first unit, and
wherein the step of performing the medical procedure comprises conducting the cryogenic fluid for the predetermined amount of time.

80. A method as recited in claim 78, wherein the step of performing the medical procedure comprises conducting a plurality of cryogenic fluids from the second unit to the first unit.

81. A method as recited in claim 80,
wherein the information stores at least one parameter indicative of a predetermined amount of time that each of the plurality of cryogenic fluids is conducted from the second unit to the first unit, and
wherein the step of performing the medical procedure comprises conducting each of the plurality of cryogenic fluids for the respective predetermined amount of time.

82. A method as recited in claim 81,
wherein the information stores at least one parameter indicative of a predetermined sequence for applying the plurality of cryogenic fluids, and
wherein the step of performing the medical procedure comprises conducting the plurality of cryogenic fluids according to the predetermined sequence.

83. A method as recited in claim 77, wherein the first unit is a single-use, disposable unit.

84. A method as recited in claim 77, wherein the information stores physical characteristics of the first unit.

85. A method as recited in claim 77, further comprising storing information in the first unit.

86. A method as recited in claim 85, wherein the step of storing information in the first unit comprises storing any of
a number of first units used to perform the medical procedure,
an identity of the medical subject,
a date of the medical procedure,
notes related to a conduct of the medical procedure.

87. A method as recited in claim 85, wherein the step of storing information in the first unit comprises storing information during a manufacturing process of the first unit.

88. A method as recited in claim 85, wherein the step of storing information in the first unit comprises storing information by a medical professional prior to performing the medical procedure.

89. A method as recited in claim 85, wherein the step of storing information in the first unit comprises the second unit storing diagnostic information in the first unit during a conduct of the medical procedure.

90. A method as recited in claim 85, wherein the step of storing information in the first unit further comprises encrypting the information.

91. A method as recited in claim 77, wherein the information comprises at least one parameter indicative of a timing of events during the medical procedure.

92. A method for performing a medical procedure, comprising:
engaging a first unit with a second unit;
conveying information stored in the first unit to the second unit; and
configuring the second unit for performing automated portions of the medical procedure responsive to the information copied from the first unit,
wherein the first unit is insertable into the body of a medical subject.

93. A method as recited in claim 92, wherein the step of configuring comprises specifying a duration of time for which a cryogenic fluid is to be conducted from the second unit to the first unit during the medical procedure.

94. A method as recited in claim 92, wherein the step of configuring comprises specifying an amount of cryogenic fluid to be conducted from the second unit to the first unit during the medical procedure.

95. A method as recited in claim 92, wherein the step of configuring comprises specifying a duration of time for which each of a plurality of cryogenic fluids are to be conducted from the second unit to the first unit during the medical procedure.

96. A method as recited in claim 92, wherein the step of configuring comprises specifying an amount of each of a plurality of cryogenic fluids to be conducted from the second unit to the first unit during the medical procedure.

97. A method as recited in claim 92, wherein the step of configuring comprises specifying a sequence for which each of a plurality of cryogenic fluids are to be conducted from the second unit to the first unit during the medical procedure.
